(19) United States
(12) Patent Application Publication
(10) Pub. No.: US 2026/0196346 A1
(43) Pub. Date: Jul. 9, 2026

(54) METHOD FOR CALCULATING EXPECTED ADULT HEIGHT AND APPARATUS FOR PERFORMING SAME

(71) Applicant: CRESCOM CO., LTD., Seongnam, Gyeonggi-do (KR)

(72) Inventor: Jae Joon LEE, Seongnam-si (KR)

(73) Assignee: CRESCOM CO., LTD., Seongnam, Gyeonggi-do (KR)

(21) Appl. No.: 19/131,699

(22) PCT Filed: Nov. 9, 2023
(86) PCT No.: PCT/KR2023/017956

§ 371 Date: May 21, 2025

(30) Foreign Application Priority Data
Nov. 29, 2022 (KR) 10-2022-0162713
Dec. 8, 2022 (KR) 10-2022-0170249

(51) Int. Cl. G16H 40/63; G06N 5/022

(52) U.S. Cl. CPC G16H 40/63; G06N 5/022

(57) ABSTRACT

A method for calculating an expected adult height, according to an embodiment of the present application, comprises the steps of: generating a growth prediction model; and calculating an expected adult height by using the generated growth prediction model, wherein the step of generating the growth prediction model further comprises the steps of: acquiring growth statistics data and input data including height information at a point in time of measurement, chronological age information at the point in time of measurement, bone age information at the point in time of measurement, and gender information; calculating the adult height on the basis of the input data and the growth statistics data; and generating the growth prediction model on the basis of the input data and the calculated adult height.

### TECHNICAL FIELD

The present application relates to growth prediction or growth analysis. Specifically, the present application relates to a method of calculating an expected adult height and an electronic device for performing the method.

### BACKGROUND ART

With the growing concern for the full and proper growth of children, growth prediction technologies for predicting a child's future height are attracting attention.

Among conventional growth prediction techniques, there is a genetic-based prediction technique for receiving parental heights reflecting genetic characteristics as inputs and predicting an adult height. However, the genetic-based prediction technique has limitations in that it does not reflect the growth development status of an analyzed person at the time of analysis, does not reflect the statistical characteristics of growth which are affected by country, sex, race, and time period, and simply calculates an adult height on the basis of parental heights.

Further, among conventional growth prediction techniques, there are bone age-based prediction techniques for predicting an adult height using a bone age. The bone age-based prediction techniques include 1) a Bayley and Pinneau (BP) technique (an adult height prediction method proposed by the researchers of the Greulich-Pyle (GP) bone age determination technique) for calculating a growth rate on the basis of a difference between a chronological age and a bone age and predicting an adult height using a current height, a current age, and a growth rate and 2) an adult height prediction (AHP) technique (an adult height prediction method proposed by the researchers of a Tanner-Whitehouse 3 (TW3) bone age technique) for calculating a predicted height of an adult for boys of ten years and older and girls of seven years and older on the basis of a current height, a Rus score based on a bone age, and a constant in accordance with a chronological age and calculating a predicted height of an adult for boys under ten years of age and girls under seven years of age on the basis of a current height, a chronological age, and a constant in accordance with the chronological age. However, the bone age-based prediction techniques are limited by their inability to reflect genetic, temporal, regional, and racial characteristics and by their relative inaccuracy in subjects who require or are undergoing growth-related treatments, such as growth hormone therapy, growth precocity therapy, and the like, outside the normal population.

Therefore, it is necessary to develop a method of calculating an expected adult height to calculate a final height of a child more precisely and a device for performing the method.

### DISCLOSURE

### Technical Problem

The present invention is directed to providing an expected adult height calculation method for calculating an expected adult height to reflect a growth development status at the time of analysis and temporal, regional, and racial characteristics and a device for performing the expected adult height calculation method.

The present invention is also directed to providing an expected adult height calculation method for calculating an expected adult height to reflect genetic characteristics and a device for performing the expected adult height calculation method.

The present invention is also directed to providing an expected adult height calculation method for calculating an expected adult height not only for a normal group but also for a group undergoing growth-related treatment and a device for performing the expected adult height calculation method.

Objects to be achieved by the present invention are not limited to those described above, and other objects which have not been described will be clearly understood by those skilled in the technical field to which the present invention pertains from the present specification and the accompanying drawings.

### Technical Solution

One aspect of the present application provides a method of calculating an expected adult height, the method including generating a growth prediction model and calculating an expected adult height using the generated growth prediction model. The generating of the growth prediction model includes acquiring growth statistics data and input data including height information at a time of measurement, chronological age information at the time of measurement, bone age information at the time of measurement, and sex information, calculating an adult height on the basis of the input data and the growth statistics data, acquiring an actual adult height of a child corresponding to the input data, and generating the growth prediction model on the basis of the input data, the calculated adult height, and the actual adult height.

Another aspect of the present application provides an electronic device including a processor configured to acquire growth statistics data and input data including height information at a time of measurement, chronological age information at the time of measurement, bone age information at the time of measurement, and sex information, calculate an adult height on the basis of the input data and the growth statistics data, acquire an actual adult height of a child corresponding to the input data, generate a growth prediction model on the basis of the input data, the calculated adult height, and the actual adult height, and calculate an expected adult height using the generated growth prediction model.

Technical solutions of the present invention are not limited to those described above, and other technical solutions which have not been described will be clearly understood by those skilled in the technical field to which the present invention pertains from the present specification and the accompanying drawings.

### Advantageous Effects

With a method of calculating an expected adult height and an electronic device for performing the method according to embodiments of the present application, it is possible to calculate an expected adult height which reflects various characteristics affecting growth, by generating a growth prediction model using adult heights calculated on the basis of growth statistics data which reflects temporal, regional, and racial characteristics.

With a method of calculating an expected adult height and an electronic device for performing the method according to embodiments of the present application, it is possible to generate a growth prediction model for each age band and/or each growth treatment group, and predict an expected adult height with higher accuracy by calculating the expected adult height using a growth prediction model corresponding to a group to which a subject of analysis belongs.

With a method of calculating an expected adult height and an electronic device for performing the method according to embodiments of the present application, it is possible to generate a growth prediction model to which genetic characteristics are applied using genetically expected heights as a dataset, and calculate an expected adult height reflecting genetic characteristics through the growth prediction model to which genetic characteristics are applied.

Effects of the present invention are not limited to those described above, and other effects which have not been described will be clearly understood by those skilled in the technical field to which the present invention pertains from the present specification and the accompanying drawings.

### DESCRIPTION OF DRAWINGS

FIG. 1 is a schematic diagram of an electronic device according to an embodiment of the present application.

FIG. 2 is a diagram illustrating operations of the electronic device according to the embodiment of the present application.

FIG. 3 is a diagram illustrating an operation of the electronic device generating a growth prediction model according to the embodiment of the present application.

FIG. 4 is a table showing an example of growth statistics data according to the embodiment of the present application.

FIG. 5 is a diagram illustrating an operation of the electronic device calculating an expected adult height using a growth prediction model according to the embodiment of the present application.

FIG. 6 is a flowchart illustrating a method of calculating an expected adult height according to the embodiment of the present application.

FIG. 7 is a flowchart specifying an operation of generating a growth prediction model according to the embodiment of the present application.

FIG. 8 is a flowchart specifying an operation of calculating an expected adult height using a growth prediction model according to the embodiment of the present application.

### BEST MODE OF THE INVENTION

A method of calculating an expected adult height according to an embodiment of the present application may include an operation of generating a growth prediction model and an operation of calculating an expected adult height using the generated growth prediction model. The operation of generating the growth prediction model may include an operation of acquiring growth statistics data and input data including height information at a time of measurement, chronological age information at the time of measurement, bone age information at the time of measurement, and sex information, an operation of calculating an adult height on the basis of the input data and the growth statistics data, an operation of acquiring an actual adult height of a child corresponding to the input data, and an operation of generating the growth prediction model on the basis of the input data, the calculated adult height, and the actual adult height.

### MODES OF THE INVENTION

The above-described objects, features, and advantages of the present application will be more obvious from the following detailed description associated with the accompanying drawings. However, the present application can be modified in various ways and have several embodiments, and specific embodiments will be illustrated in the drawings and described in detail below.

Throughout the specification, like reference numerals refer to like components in principle. Also, components having the same function within the scope of the same idea illustrated in the drawings of embodiments will be referred to with the same reference numerals, and reiterative description thereof will be omitted.

When detailed description of a known function or element related to the present application is determined to unnecessarily obscure the subject matter of the present application, the detailed description will be omitted. Also, numbers (e.g., first, second, and the like) used in the description process of the present specification are merely identification code for distinguishing one component from others.

In addition, the terms “module” and “unit” for components used in the following embodiments are given or used together only to facilitate the writing of the specification. Therefore, these terms do not have distinct meanings or roles therein.

In the following embodiments, singular forms include plural forms unless the context clearly indicates otherwise.

In the following embodiments, the term “include,” “have” or the like means that a feature or component described in the specification is present, and does not preclude the possibility that one or more other features or components are added.

For convenience of description, the sizes of components may be exaggerated or reduced in the drawings. For example, the size and thickness of each component illustrated in the drawings are arbitrarily indicated for convenience of description, and the present invention is not necessarily limited to what is illustrated.

When an embodiment can be implemented differently, a specific process may be performed in a different order from the described order of the process. For example, two processes described in succession may be performed substantially simultaneously, or may be performed in a reverse order to the described order.

In the following embodiments, when components and the like are referred to as being connected, the components may not only be directly connected but may also be indirectly connected with a component and the like interposed therebetween.

For example, in the present specification, when components and the like are referred to as being electrically connected, the components may not only be directly electrically connected but may also be indirectly electrically connected with a component and the like interposed therebetween.

A method of calculating an expected adult height according to an embodiment of the present application may include an operation of generating a growth prediction model and an operation of calculating an expected adult height using the generated growth prediction model. The operation of generating the growth prediction model may further include an operation of acquiring growth statistics data and input data including height information at a time of measurement, chronological age information at the time of measurement, bone age information at the time of measurement, and sex information, an operation of calculating an adult height on the basis of the input data and the growth statistics data, an operation of acquiring an actual adult height of a child corresponding to the input data, and an operation of generating the growth prediction model on the basis of the input data, the calculated adult height, and the actual adult height.

According to an embodiment of the present application, the operation of calculating the adult height may further include an operation of acquiring a first percentile corresponding to the chronological age information and the height information of the input data from the growth statistics data on the basis of the height information and the chronological age information and an operation of calculating a first height corresponding to the first percentile as the adult height using growth statistics data of a group of people whose growth has stopped.

According to an embodiment of the present application, the operation of calculating the adult height may further include an operation of acquiring a second percentile corresponding to the bone age information and the height information of the input data from the growth statistics data on the basis of the height information and the bone age information and an operation of calculating a second height corresponding to the second percentile as the adult height using growth statistics data of a group of people whose growth has stopped.

According to an embodiment of the present application, the operation of generating the growth prediction model may further include an operation of inputting the height information, the chronological age information, the bone age information, and the sex information of the input data and the calculated adult height as input values for the growth prediction model, an operation of inputting the actual adult height as an output value of the growth prediction model, and an operation of calculating a weight of the growth prediction model for outputting the output value from the input values.

According to an embodiment of the present application, the operation of calculating the expected adult height may further include an operation of acquiring subject input data including height information at a time of analysis, chronological age information at the time of analysis, bone age information at the time of analysis, and sex information regarding a subject whose expected adult height will be analyzed and growth statistics data of a group to which the subject belongs and an operation of calculating the expected adult height from the subject input data using the growth prediction model including the calculated weight.

According to an embodiment of the present application, the operation of calculating the expected adult height may further include an operation of calculating an adult height from the subject input data of the subject on the basis of the growth statistics data and an operation of inputting the subject input data and the calculated adult height into the growth prediction model and acquiring the expected adult height output from the growth prediction model.

According to an embodiment of the present application, the operation of generating the growth prediction model may further include an operation of, when an identified age band corresponds to a first band, generating a first model including a first weight on the basis of a dataset composed of adult heights which are calculated from the growth statistics data and input data corresponding to the first band, and the actual adult height, and an operation of, when the identified age band corresponds to a second band, generating a second model including a second weight on the basis of a dataset of adult heights which are calculated from the growth statistics data and input data corresponding to the second band, and the actual adult height.

According to an embodiment of the present application, the operation of calculating the expected adult height may further include an operation of identifying a preset age band on the basis of the chronological age information or the bone age information of the subject input data and an operation of, when the identified age band corresponds to the first band, calculating the expected adult height from the adult height calculated from the subject input data and the growth statistics data using the first model, and an operation of, when the identified age band corresponds to the second band, generating the expected adult height from the adult height calculated from the subject input data and the growth statistics data using the second model.

According to an embodiment of the present application, a computer-readable recording medium on which a program for executing the method of calculating an expected adult height is recorded may be provided.

An electronic device according to an embodiment of the present application may include a processor configured to acquire growth statistics data and input data including height information at a time of measurement, chronological age information at the time of measurement, bone age information at the time of measurement, and sex information, calculate an adult height on the basis of the input data and the growth statistics data, acquire an actual adult height of a child corresponding to the input data, generate a growth prediction model on the basis of the input data, the calculated adult height, and the actual adult height, and calculate an expected adult height using the generated growth prediction model.

Hereinafter, an expected adult height calculation method of the present application and an electronic device (or a server, hereinafter, “electronic device”) for performing the method will be described.

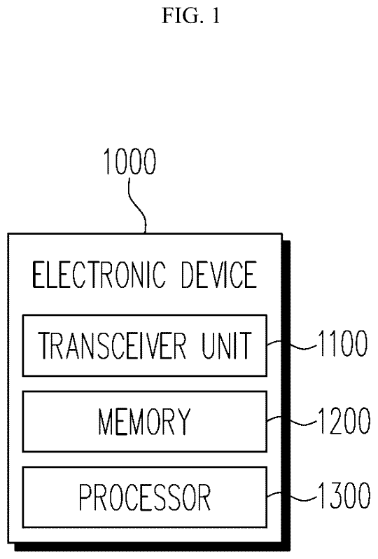

FIG. 1 is a schematic diagram of an electronic device 1000 according to an embodiment of the present application.

The electronic device 1000 according to the embodiment of the present application may include a transceiver unit 1100, a memory 1200, and a processor 1300.

The transceiver unit 1100 of the electronic device 1000 may communicate with any external device (or external server) including a user terminal and/or a database. For example, the electronic device 1000 may acquire an input that requests calculation of an expected adult height from the user terminal through the transceiver unit 1100. For example, the electronic device 1000 may acquire any data that is required for calculating an expected adult height and includes height information at the time of analysis, chronological age information at the time of analysis, bone age information at the time of analysis, and/or sex information regarding a subject whose expected adult height will be analyzed, from the user terminal through the transceiver unit 1100. For example, the electronic device 1000 may acquire growth statistics data that is classified by age, sex, country, region, and/or race from the database through the transceiver unit 1100. For example, the electronic device 1000 may transmit the calculated adult height information to any external device including the user terminal through the transceiver unit 1100.

The electronic device 1000 may connect to a network through the transceiver unit 1100 to transmit and receive various data. The transceiver unit 1100 may be a wired type transceiver unit or a wireless type transceiver unit. Since the wired type transceiver unit and the wireless type transceiver unit each have advantages and disadvantages, the electronic device 1000 may be provided with both the wired type transceiver unit and the wireless type transceiver unit in some cases. Here, the wireless type transceiver unit may mainly use a wireless local area network (WLAN)-based communication scheme such as Wi-Fi. Alternatively, the wireless type transceiver unit may use a cellular communication scheme, for example, a Long-Term Evolution (LTE) or fifth generation (5G) communication scheme. However, a wireless communication protocol is not limited to the above examples, and it is possible to use any appropriate wireless-type communication scheme. Representative examples of the wired type transceiver unit employ LAN and Universal Serial Bus (USB) communication, and other communication schemes are also available.

The memory 1200 of the electronic device 1000 may store various information. Various data may be temporarily or semi-temporarily stored in the memory 1200. Examples of the memory 1200 may be a hard disk drive (HDD), a solid state drive (SSD), a flash memory, a read-only memory (ROM), a random access memory (RAM), and the like. The memory 1200 may be provided in a form that is embedded into or detachable from the electronic device 1000. Various data necessary for operations of the electronic device 1000, such as an operating system (OS) for operating the electronic device 1000 and a program for operating each element of the electronic device 1000, may be stored in the memory 1200.

The processor 1300 may control overall operations of the electronic device 1000. For example, the processor 1300 may control overall operations of the electronic device 1000, which include an operation of generating a growth prediction model, which will be described below, and/or an operation of calculating an expected adult height using the growth prediction model. Specifically, the processor 1300 may load a program for the overall operations of the electronic device 1000 from the memory 1200 and execute the program. The processor 1300 may be implemented as an application processor (AP), a central processing unit (CPU), a microcontroller unit (MCU), or a similar device in accordance with hardware, software, or a combination thereof. As hardware, the processor 1300 may be provided in the form of an electronic circuit for processing an electrical signal to perform a control function, and as software, may be provided in the form of a program or code for operating a hardware circuit.

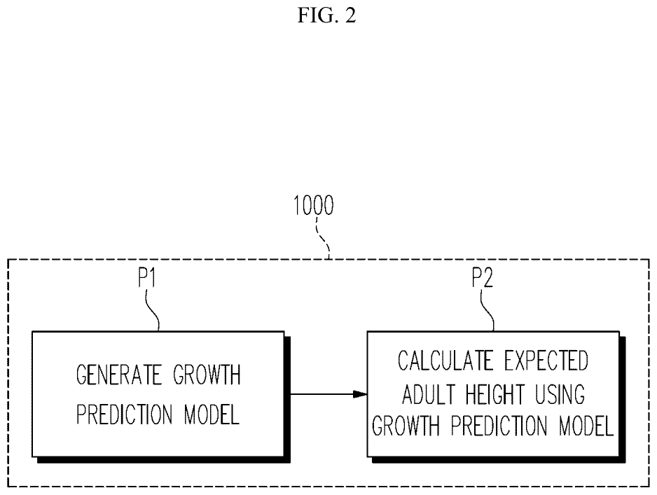

Operations of the electronic device 1000 according to the embodiment of the present application and a method of calculating an expected adult height performed by the electronic device 1000 will be described in detail below with reference to FIGS. 2 to 8.

FIG. 2 is a diagram illustrating operations of the electronic device 1000 according to the embodiment of the present application.

The electronic device 1000 according to the embodiment of the present application may generate a growth prediction model for predicting an expected adult height. Further, the electronic device 1000 may be configured to calculate an expected adult height from subject input data of a subject of analysis including height information at the time of analysis, age information (e.g., a bone age and a chronological age), and/or sex information using the generated growth prediction model.

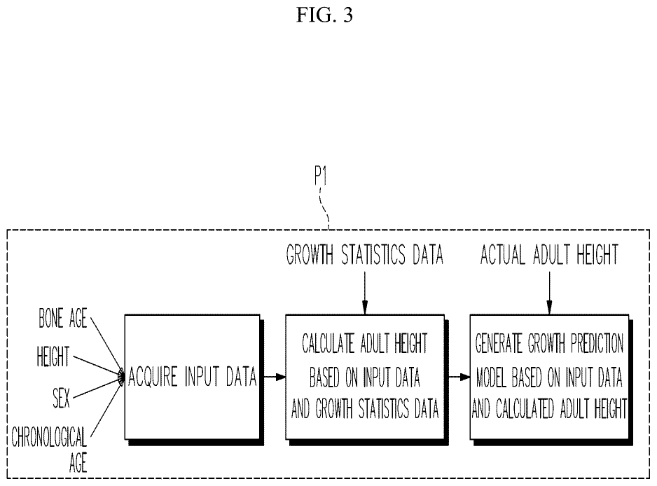

FIG. 3 is a diagram illustrating an operation of the electronic device 1000 generating a growth prediction model according to the embodiment of the present application.

The electronic device 1000 according to the embodiment of the present application may acquire input data through the transceiver unit 1100. The input data may include any information that may affect an adult height including height information of a specific time point, age information (e.g., bone age information and chronological age information), sex information, parental height information, and/or growth treatment information.

Further, the electronic device 1000 according to the embodiment of the present application may acquire growth statistics data through the transceiver unit 1100.

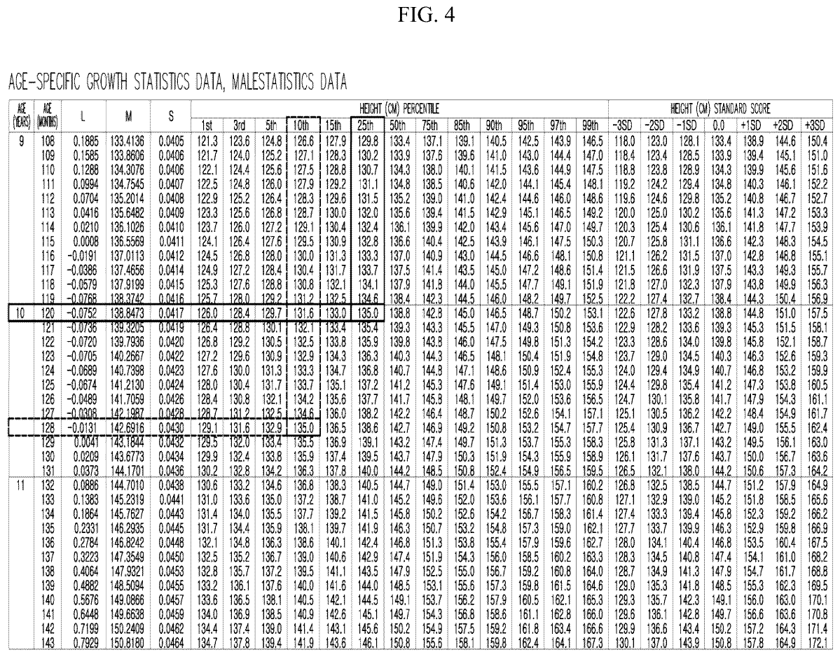

FIG. 4 is a table showing an example of growth statistics data according to the embodiment of the present application.

Growth statistics data may be grouped by age, sex, country, region, and/or race, and grouped growth statistics data may include statistics data related to a height percentile in accordance with ages (e.g., bone ages or chronological ages) of a corresponding group.

The electronic device 1000 according to the embodiment of the present application may calculate an adult height on the basis of the input data and the growth statistics data.

As an example, the electronic device 1000 may acquire a first percentile corresponding to the height information and the chronological age information of the input data from the growth statistics data on the basis of the height information and the chronological age information. For example, when the chronological age information of the input data is 10 years of age 120 months and the height information of the input data is 135.0 cm, the electronic device 1000 may acquire a first percentile (a height percentile of 25 in FIG. 4) corresponding to the chronological age information (10 years of age 120 months in FIG. 4) and the height information (135.0 cm in FIG. 4) using growth statistics data corresponding to the sex information of the input data. Further, the electronic device 1000 may calculate, as the adult height, a first height corresponding to the first percentile using growth statistics data of a group of people whose growth has stopped (e.g., growth statistics data of a preset age). For example, the electronic device 1000 may calculate, as the adult height, a first height corresponding to the first percentile (e.g., the height percentile of 25 acquired as described above) using the group of people whose growth has stopped (e.g., growth statistics data of an adult age group).

As an example, the electronic device 1000 may acquire a second percentile corresponding to the bone age information and the height information of the input data from the growth statistics data on the basis of the height information and the bone age information. For example, when the bone age information of the input data is 10 years of age 128 months and the height information of the input data is 135.0 cm, the electronic device 1000 may acquire a second percentile (a height percentile of 10 in FIG. 4) corresponding to the bone age information (10 years of age 128 months in FIG. 4) and the height information (135.0 cm in FIG. 4) using the growth statistics data corresponding to the sex information of the input data. Further, the electronic device 1000 may calculate, as the adult height, a second height corresponding to the second percentile using growth statistics data of a group of people whose growth has stopped (e.g., growth statistics data of a preset age). For example, the electronic device 1000 may calculate, as the adult height, a second height corresponding to the second percentile (e.g., the height percentile of 10 acquired as described above) using the group of people whose growth has stopped (e.g., growth statistics data of an adult age group).

As an example, the electronic device 1000 may calculate the adult height on the basis of the foregoing first height calculated using the chronological age information and the foregoing second height calculated using the bone age information. For example, the electronic device 1000 may assign a first weight to the first height, assign a second weight to the second height, and calculate the adult height on the basis of the first height to which the first weight has been assigned and the second height to which the second weight has been assigned. For example, the electronic device 1000 may be implemented to calculate a weighted sum of the first height and the second height as the adult height.

As an example, the electronic device 1000 may be implemented to calculate the adult height on the basis of a weighted sum of the bone age information and the chronological age information of the input data. For example, when the bone age information of the input data is 12 years of age and the chronological information is 10 years of age, the electronic device 1000 may acquire weighted average age information corresponding to a weighted average of the two values and acquire a percentile from the growth statistics data using the weighted average age information and the height information of the input data. For example, when the two values have the same weight, the electronic device 1000 may calculate a third percentile corresponding to weighted average age information including a value of 11-year-old children and the height information of the input data using the growth statistics data. Here, the electronic device 1000 may calculate, as the adult height, a third height corresponding to the third percentile using a group of people whose growth has stopped (e.g., growth statistics data of a preset age), which is similar to the above description.

However, the above description of adult height calculation is merely illustrative, and the electronic device 1000 may be implemented to calculate the adult height from the input data by utilizing any appropriate method.

Further, the electronic device 1000 according to the embodiment of the present application may generate a growth prediction model on the basis of the input data and the calculated adult height. More specifically, the electronic device 1000 may acquire an actual adult height of a child corresponding to the input data. Here, the electronic device 1000 may generate the growth prediction model on the basis of the input data, the calculated adult height, and/or the actual adult height (ground truth).

As an example, the electronic device 1000 may generate the growth prediction model by utilizing a regression analysis technique. Specifically, the electronic device 1000 may generate the growth prediction model including optimal weights using the following expression.

$${y}^{k} = {\sum }_{i}^{m} {w}_{i} {x}_{i}^{k} + b \qquad [ Expression ]$$

k: A k^{th} dataset composed of an actual adult height and dependent variables (values of information included in input data and/or an adult height calculated from growth statistics data)
y^{k}: An actual adult height of the k^{th} dataset

$${x}_{i}^{k} :$$

An input value i (a chronological age, a bone age, a height (a child height and a calculated adult height), a sex, and the like) of the k^{th} dataset
wᵢ: A weight for the input value i

Specifically, the electronic device 1000 may be implemented to input the adult height calculated from the height information (e.g., child height information), the chronological age information, the bone age information, and/or the sex information of the input data and/or the growth statistics data as an input value x for the growth prediction model and input the actual adult height as an output value y of the growth prediction model. Here, the electronic device 1000 may calculate a weight w of the growth prediction model for outputting the output value from the input value.

For example, the electronic device 1000 may generate the growth prediction model by utilizing a machine learning technique. Specifically, the electronic device 1000 may train the growth prediction model using a training dataset including input data which includes height information, chronological age information, bone age information, sex information, and/or adult heights calculated from the growth statistics data, and label information composed of actual adult heights. More specifically, the electronic device 1000 may input an adult height which is calculated from the height information, the chronological age information, the bone age information, the sex information, and/or the growth statistics data into the growth prediction model, acquire output values output from the growth prediction model, and update parameters included in the growth prediction model on the basis of differences between the output values and the actual adult heights included in the label information, thereby training the growth prediction model. For example, the electronic device 1000 may update the parameters of the growth prediction model such that the output values of the growth prediction model may approximate to the actual adult heights included in the label information.

Meanwhile, the electronic device 1000 according to the embodiment of the present application may generate a growth prediction model per age band. Specifically, the electronic device 1000 may identify a preset age band on the basis of the age information (e.g., the chronological age information or the bone age information) of the input data. For example, preset age bands may be previously set as a pre-pubescent age band and a post-pubescent age band. In the case of female children, the preset age bands may be previously set as an age band of 4 years to 8 years and an age band of 8 years or more.

Here, when the identified age band corresponds to a first band, the electronic device 1000 may generate a first model including a first weight as described above on the basis of a dataset composed of adult heights calculated from the growth statistics data and input data corresponding to the first band, and the actual adult heights. On the other hand, when the identified age band corresponds to a second band, the electronic device 1000 may generate a second model including a second weight as described above on the basis of a dataset composed of adult heights calculated from the growth statistics data and input data corresponding to the second band, and the actual adult heights. In other words, the electronic device 1000 may generate an optimized growth prediction model per age band.

Meanwhile, the foregoing preset age bands are examples for illustrative purposes only, and any appropriate age bands may be set.

Meanwhile, although not shown in FIG. 3, the electronic device 1000 may acquire parental height information as input data to calculate an expected adult height on the basis of genetic factors. For example, the electronic device 1000 may acquire a father's height information and a mother's height information and calculate a genetically expected height using the following genetically expected height expression, acquiring the genetically expected height as input data. Further, the electronic device 1000 may be implemented to input the calculated genetically expected height as an input value x of a dataset for the growth prediction model.

### Expression of Genetically Expected Height

$$Male child \text{:} ( father ' s height + mother ' s height ) / 2 + 6.5 cm$$

$$Female child \text{:} ( father ' s height + mother ' s height ) / 2 - 6.5 cm$$

Weights (or parameters) of the growth prediction model calculated by inputting the genetically expected height as the dataset can reflect genetic characteristics, and the growth prediction model including the weights (or parameters) reflecting the genetic characteristics can provide an effect of calculating an expected adult height on the basis of the genetic characteristics.

Further, the electronic device 1000 may generate a growth prediction model per growth treatment group. Specifically, the electronic device 1000 may acquire a group identifier for identifying a growth treatment group as input data. For example, the electronic device 1000 may acquire, as input data, a group identifier that indicates whether the input data corresponds to a normal group, a growth hormone treatment group, a precocious puberty suppression treatment group, and/or a growth hormone-precocious puberty suppression treatment group. Here, the electronic device 1000 may generate a growth prediction model per group on the basis of the group identifier. Specifically, when the input data includes a first group identifier (e.g., an identifier indicating the normal group), the electronic device 1000 may generate a third model including a third weight for a first group on the basis of the input data (e.g., a dataset related to the normal group), adult heights calculated from the growth statistics data, and/or the actual adult heights. On the other hand, when the input data includes a second group identifier (e.g., an identifier indicating any one of the growth hormone treatment group, the precocious puberty suppression treatment group, and/or the growth hormone-precocious puberty suppression treatment group), the electronic device 1000 may generate a fourth model including a fourth weight for a second group on the basis of the input data (e.g., a dataset related to the treatment group), adult heights calculated from the growth statistics data, and/or the actual adult heights.

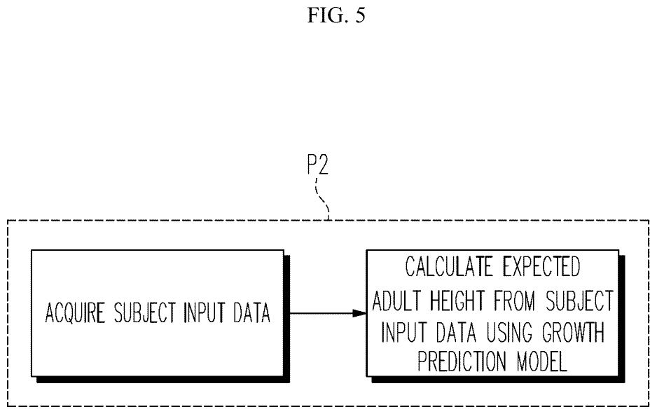

FIG. 5 is a diagram illustrating an operation of the electronic device 100 calculating an expected adult height using a growth prediction model according to the embodiment of the present application.

The electronic device 1000 according to the embodiment of the present application may acquire subject input data including height information at a time of analysis, chronological age information at the time of analysis, bone age information at the time of analysis, and/or sex information regarding a subject whose expected adult height will be analyzed, through the transceiver unit 1100. Further, the electronic device 1000 may acquire growth statistics data through the transceiver unit 1100. For example, the electronic device 1000 may acquire growth statistics data corresponding to a group to which the subject belongs (e.g., a group corresponding to the subject's sex, age, and/or treatment information) through the transceiver unit 1100.

Further, the electronic device 1000 according to the embodiment of the present application may be implemented to calculate an adult height from the subject input data including the subject's height information, chronological age information, bone age information, and/or sex information on the basis of the growth statistics data.

For example, the electronic device 1000 may acquire a percentile corresponding to the chronological age information and the height information of the subject input data from the growth statistics data on the basis of the height information and the chronological age information. Further, the electronic device 1000 may calculate a height corresponding to the percentile as the adult height using growth statistics data of a group of people whose growth has stopped (e.g., growth statistics data of a preset age).

For example, the electronic device 1000 may acquire a percentile corresponding to the bone age information and the height information of the subject input data from the growth statistics data on the basis of the height information and the bone age information. Further, the electronic device 1000 may calculate a height corresponding to the percentile as the adult height using growth statistics data of a group of people whose growth has stopped (e.g., growth statistics data of a preset age).

For example, the electronic device 1000 may acquire a percentile corresponding to the height information and weighted average age information of the bone age information and the chronological age information of the subject input data from the growth statistics data on the basis of the weighted average age information. Further, the electronic device 1000 may calculate a height corresponding to the percentile as the adult height using growth statistics data of a group of people whose growth has stopped (e.g., growth statistics data of a preset age).

Further, the electronic device 1000 according to the embodiment of the present application may calculate an expected adult height from the adult height calculated from the subject input data and/or the growth statistics data using the generated growth prediction model. Specifically, the electronic device 1000 may calculate the expected adult height from an adult height calculated from the subject input data and/or the growth statistics data using the growth prediction model that includes weights (or parameters) derived by an artificial intelligence model as described above. For example, the electronic device 1000 may input the adult height which is calculated from the height information, the chronological age information, the bone age information, and/or the sex information of the subject input data and/or the growth statistics data, into the generated growth prediction model, and acquire the expected adult height on the basis of calculated weights (or updated parameters) of the generated growth prediction model.

Further, the electronic device 1000 according to the embodiment of the present application may be implemented to calculate the expected adult height using a growth prediction model that is generated per age band. Specifically, the electronic device 1000 may identify a preset age band on the basis of the age information (e.g., the chronological age information or the bone age information) of the subject input data. Here, when the identified age band corresponds to the first band (e.g., the pre-pubescent age band), the electronic device 1000 may calculate the expected adult height from the adult height which is calculated from the subject input data and/or the growth statistics data using the first model including the first weight. On the other hand, when the identified age band corresponds to the second band (e.g., the post-pubescent age band), the electronic device 1000 may calculate the expected adult height from the adult height which is calculated from the subject input data and/or the growth statistics data using the second model including the second weight.

Further, the electronic device 1000 according to the embodiment of the present application may be implemented to calculate the expected adult height using a growth prediction model that is generated per treatment group. Specifically, the electronic device 1000 may acquire, as subject input data, a group identifier that indicates whether the subject input data corresponds to a normal group, a growth hormone treatment group, a precocious puberty suppression treatment group, and/or a growth hormone-precocious puberty suppression treatment group. Here, the electronic device 1000 may select a model for calculating an expected adult height on the basis of the group identifier and calculate the expected adult height from the adult height calculated from the bone age information, the chronological age information, the height information, and/or the sex information of the subject input data and/or the growth statistics data. For example, when the subject input data includes the first group identifier (e.g., the identifier indicating the normal group), the electronic device 1000 may input the adult height calculated from the subject input data and/or the growth statistics data into the third model including the third weight, thereby acquiring the expected adult height. For example, when the subject input data includes the second group identifier (e.g., the identifier indicating any one of the growth hormone treatment group, the precocious puberty suppression treatment group, and/or the growth hormone-precocious puberty suppression treatment group), the electronic device 1000 may input the adult height calculated from the subject input data and/or the growth statistics data into the fourth model including the fourth weight, thereby acquiring the expected adult height.

Meanwhile, although not described in detail for convenience of description, the electronic device 1000 may be implemented to generate a growth prediction model for each of the growth hormone treatment group, the precocious puberty suppression treatment group, and/or the growth hormone-precocious puberty suppression treatment group and calculate an optimized expected adult height for each group using the growth prediction model generated for the group.

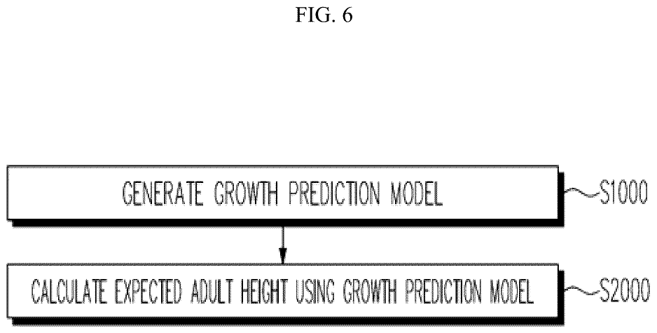

A method of calculating an expected adult height according to an embodiment of the present application will be described in further detail below with reference to FIGS. 6 to 8. In describing the method of calculating an expected adult height, embodiments overlapping the description of FIGS. 2 to 5 may be omitted. However, this is for convenience of description, and the present application is not limited by the omission.

FIG. 6 is a flowchart illustrating a method of calculating an expected adult height according to an embodiment of the present application.

The method of calculating an expected adult height according to the embodiment of the present application may include an operation S1000 of generating a growth prediction model and an operation S2000 of calculating an expected adult height using the growth prediction model.

In the operation S1000 of generating a growth prediction model, the electronic device 1000 may generate a growth prediction model for predicting an expected adult height on the basis of age information (e.g., bone age information or chronological age information), height information, and/or sex information.

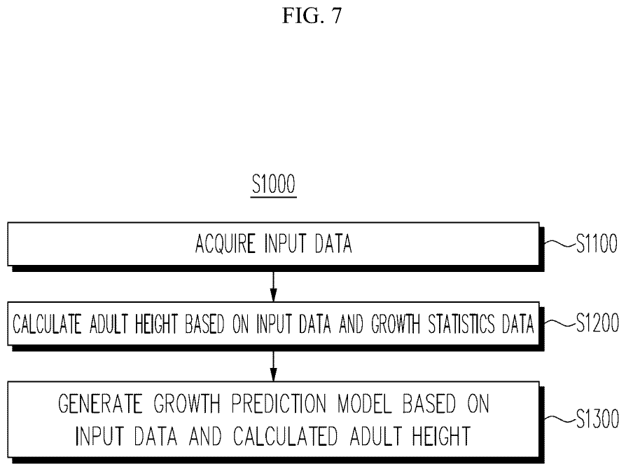

FIG. 7 is a flowchart specifying the operation S1000 of generating a growth prediction model according to the embodiment of the present application.

The operation S1000 of generating a growth prediction model according to the embodiment of the present application may further include an operation S1100 of acquiring input data, an operation S1200 of calculating an adult height on the basis of the input data and growth statistics data, and an operation S1300 of generating a growth prediction model on the basis of the input data and the calculated adult height.

In the operation S1100 of acquiring input data, the electronic device 1000 may acquire input data through the transceiver unit 1100. The input data may include any information that may affect an adult height including height information of a specific time point, age information (e.g., bone age information and chronological age information), sex information, parental height information, and/or growth treatment information.

Further, in the operation S1100 of acquiring input data, the electronic device 1000 may acquire growth statistics data through the transceiver unit 1100. The growth statistics data may be grouped by age, sex, country, region, and/or race, and grouped growth statistics data may include statistics data related to a height percentile in accordance with ages of a corresponding group.

In the operation S1200 of calculating an adult height on the basis of the input data and the growth statistics data, the electronic device 1000 may calculate an adult height on the basis of the input data and the growth statistics data.

As an example, the electronic device 1000 may acquire a first percentile corresponding to the height information and the chronological age information of the input data from the growth statistics data on the basis of the height information and the chronological age information. For example, when the chronological age information of the input data is 10 years of age 120 months and the height information of the input data is 135.0 cm, the electronic device 1000 may acquire a first percentile (a height percentile of 25 in FIG. 4) corresponding to the chronological age information (10 years of age 120 months in FIG. 4) and the height information (135.0 cm in FIG. 4) using growth statistics data corresponding to the sex information of the input data. Further, the electronic device 1000 may calculate, as the adult height, a first height corresponding to the first percentile using growth statistics data of a group of people whose growth has stopped (e.g., growth statistics data of a preset age). For example, the electronic device 1000 may calculate, as the adult height, a first height corresponding to the first percentile (e.g., the height percentile of 25 acquired as described above) using the group of people whose growth has stopped (e.g., growth statistics data of an adult age group).

As an example, the electronic device 1000 may acquire a second percentile corresponding to the bone age information and the height information of the input data from the growth statistics data on the basis of the height information and the bone age information. For example, when the bone age information of the input data is 10 years of age 128 months and the height information of the input data is 135.0 cm, the electronic device 1000 may acquire a second percentile (a height percentile of 10 in FIG. 4) corresponding to the bone age information (10 years of age 128 months in FIG. 4) and the height information (135.0 cm in FIG. 4) using the growth statistics data corresponding to the sex information of the input data. Further, the electronic device 1000 may calculate, as the adult height, a second height corresponding to the second percentile using growth statistics data of a group of people whose growth has stopped (e.g., growth statistics data of a preset age). For example, the electronic device 1000 may calculate, as the adult height, a second height corresponding to the second percentile (e.g., the height percentile of 10 acquired as described above) using the group of people whose growth has stopped (e.g., growth statistics data of an adult age group).

As an example, the electronic device 1000 may calculate the adult height on the basis of the foregoing first height calculated using the chronological age information and the foregoing second height calculated using the bone age information. For example, the electronic device 1000 may assign a first weight to the first height, assign a second weight to the second height, and calculate the adult height on the basis of the first height to which the first weight has been assigned and the second height to which the second weight has been assigned. For example, the electronic device 1000 may be implemented to calculate a weighted sum of the first height and the second height as the adult height.

As an example, the electronic device 1000 may be implemented to calculate the adult height on the basis of a weighted sum of the bone age information and the chronological age information of the input data. For example, when the bone age information of the input data is 12 years of age and the chronological information is 10 years of age, the electronic device 1000 may acquire weighted average age information corresponding to a weighted average of the two values and acquire a percentile from the growth statistics data using the weighted average age information and the height information of the input data. For example, when the two values have the same weight, the electronic device 1000 may calculate a third percentile corresponding to weighted average age information including a value of 11-year-old children and the height information of the input data using the growth statistics data. Here, the electronic device 1000 may calculate, as the adult height, a third height corresponding to the third percentile using a group of people whose growth has stopped (e.g., growth statistics data of a preset age), which is similar to the above description.

In the operation S1300 of generating a growth prediction model on the basis of the input data and the calculated adult height, the electronic device 1000 may generate the growth prediction model on the basis of the input data and the calculated adult height. More specifically, the electronic device 1000 may acquire an actual adult height of a child corresponding to the input data. Here, the electronic device 1000 may generate the growth prediction model on the basis of the input data, the adult height calculated from the growth statistics data, and/or the actual adult height.

As an example, the electronic device 1000 may generate the growth prediction model by utilizing a regression analysis technique. Specifically, the electronic device 1000 may generate the growth prediction model including optimal weights using the following expression.

$${y}^{k} = {\sum }_{i}^{m} {w}_{i} {x}_{i}^{k} + b \qquad [ Expression ]$$

k: A k^{th} dataset composed of an actual adult height and dependent variables (values of information included in input data and/or an adult height calculated from growth statistics data)
y^{k}: An actual adult height of the k^{th} dataset

$${x}_{i}^{k} :$$

An input value i (a chronological age, a bone age, a height (a child height and a calculated adult height), a sex, and the like) of the k^{th} dataset
wᵢ: A weight for the input value i

Specifically, the electronic device 1000 may be implemented to input the adult height calculated from the height information (e.g., child height information), the chronological age information, the bone age information, and/or the sex information of the input data and/or the growth statistics data as an input value x for the growth prediction model and input the actual adult height as an output value y of the growth prediction model. Here, the electronic device 1000 may be configured to calculate a weight w of the growth prediction model for outputting the output value from the input value.

For example, the electronic device 1000 may generate the growth prediction model by utilizing a machine learning technique. Specifically, the electronic device 1000 may train the growth prediction model using a training dataset including input data which includes height information, chronological age information, bone age information, sex information, and/or adult heights calculated from the growth statistics data, and label information composed of actual adult heights. More specifically, the electronic device 1000 may input an adult height which is calculated from the height information, the chronological age information, the bone age information, the sex information, and/or the growth statistics data into the growth prediction model, acquire output values output from the growth prediction model, and update parameters included in the growth prediction model on the basis of differences between the output values and the actual adult heights included in the label information, thereby training the growth prediction model. For example, the electronic device 1000 may update the parameters of the growth prediction model such that the output values of the growth prediction model may approximate to the actual adult heights included in the label information.

Meanwhile, the electronic device 1000 according to the embodiment of the present application may generate a growth prediction model per age band. Specifically, the electronic device 1000 may identify a preset age band on the basis of the age information (e.g., the chronological age information or the bone age information) of the input data. Here, when the identified age band corresponds to a first band, the electronic device 1000 may generate a first model including a first weight as described above on the basis of a dataset composed of adult heights calculated from the growth statistics data and input data corresponding to the first band, and the actual adult heights. On the other hand, when the identified age band corresponds to a second band, the electronic device 1000 may generate a second model including a second weight as described above on the basis of a dataset composed of adult heights calculated from the growth statistics data and input data corresponding to the second band, and the actual adult heights.

In the operation S2000 of calculating an expected adult height using the growth prediction model, the electronic device 1000 may calculate an expected adult height from subject input data including height information at a time of analysis, age information (e.g., a bone age and a chronological age) at the time of analysis, and/or sex information regarding a subject of analysis using the growth prediction model generated through the operation S1000.

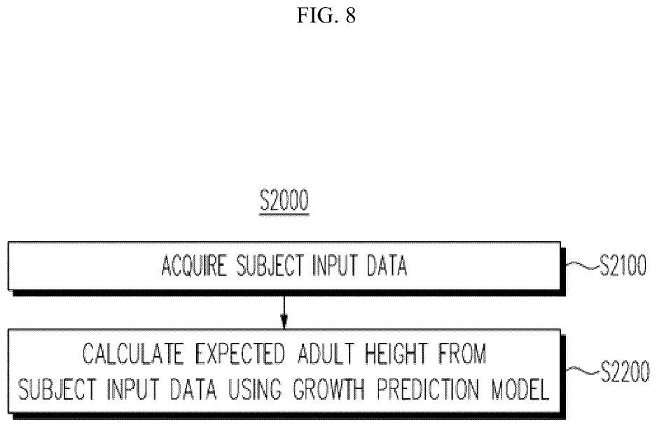

FIG. 8 is a flowchart specifying the operation S2000 of calculating an expected adult height using a growth prediction model according to an embodiment of the present application.

The operation S2000 of calculating an expected adult height using a growth prediction model according to the embodiment of the present application may further include an operation S2100 of acquiring subject input data and an operation S2200 of calculating an expected adult height from the subject input data using the growth prediction model.

In the operation S2100 of acquiring subject input data, the electronic device 1000 may acquire subject input data including the height information at the time of analysis, the chronological age information at the time of analysis, the bone age information at the time of analysis, and/or the sex information regarding the subject whose expected adult height will be analyzed, through the transceiver unit 1100.

Further, in the operation S2100 of acquiring subject input data, the electronic device 1000 may acquire growth statistics data through the transceiver unit 1100. For example, the electronic device 1000 may acquire growth statistics data corresponding to a group to which the subject belongs (e.g., a group corresponding to the subject's sex, age, and/or treatment information) through the transceiver unit 1100.

In the operation S2200 of calculating an expected adult height from the subject input data using the growth prediction model, the electronic device 1000 may be implemented to calculate the adult height from the subject's height information, chronological age information, bone age information, and/or sex information on the basis of the growth statistics data.

For example, the electronic device 1000 may acquire a percentile corresponding to the chronological age information and the height information of the subject input data from the growth statistics data on the basis of the height information and the chronological age information. Further, the electronic device 1000 may calculate a height corresponding to the percentile as the adult height using growth statistics data of a group of people whose growth has stopped (e.g., growth statistics data of a preset age).

For example, the electronic device 1000 may acquire a percentile corresponding to the bone age information and the height information of the subject input data from the growth statistics data on the basis of the height information and the bone age information. Further, the electronic device 1000 may calculate a height corresponding to the percentile as the adult height using growth statistics data of a group of people whose growth has stopped (e.g., growth statistics data of a preset age).

For example, the electronic device 1000 may acquire a percentile corresponding to the height information and weighted average age information of the bone age information and the chronological age information of the subject input data from the growth statistics data on the basis of the weighted average age information. Further, the electronic device 1000 may calculate a height corresponding to the percentile as the adult height using growth statistics data of a group of people whose growth has stopped (e.g., growth statistics data of a preset age).

Further, in the operation S2200 of calculating an expected adult height from the subject input data using the growth prediction model, the electronic device 1000 may calculate an expected adult height from the adult height calculated from the subject input data and/or the growth statistics data using the generated growth prediction model including the weights (or parameters) calculated through the operation S1300. For example, the electronic device 1000 may input the adult height which is calculated from the height information, the chronological age information, the bone age information, and/or the sex information of the subject input data and/or the growth statistics data, into the generated growth prediction model, and acquire the calculated expected adult height on the basis of the calculated weights (or updated parameters) of the generated growth prediction model.

In the operation S2200 of calculating an expected adult height from the subject input data using the growth prediction model, the electronic device 1000 may be implemented to calculate the expected adult height using a growth prediction model that is generated per age band. Specifically, the electronic device 1000 may identify a preset age band on the basis of the age information (e.g., the chronological age information or the bone age information) of the subject input data. Here, when the identified age band corresponds to the first band (e.g., a pre-pubescent age band), the electronic device 1000 may calculate the expected adult height from the adult height which is calculated from the subject input data and/or the growth statistics data using the first model including the first weight. On the other hand, when the identified age band corresponds to the second band (e.g., the post-pubescent age band), the electronic device 1000 may calculate the expected adult height from the adult height which is calculated from the subject input data and/or the growth statistics data using a second model including the second weight.

With a method of calculating an expected adult height and an electronic device for performing the method according to embodiments of the present application, it is possible to calculate an expected adult height which reflects various characteristics affecting growth, by generating a growth prediction model using adult heights calculated on the basis of growth statistics data which reflects temporal, regional, and racial characteristics.

With a method of calculating an expected adult height and an electronic device for performing the method according to embodiments of the present application, it is possible to generate a growth prediction model for each age band and/or each growth treatment group, and predict an expected adult height with higher accuracy by calculating the expected adult age using a growth prediction model corresponding to a group to which a subject of analysis belongs.

With a method of calculating an expected adult height and an electronic device for performing the method according to embodiments of the present application, it is possible to generate a growth prediction model to which genetic characteristics are applied using genetically expected heights as a dataset, and calculate an expected adult height reflecting genetic characteristics through the growth prediction model to which genetic characteristics are applied.

Various operations of the electronic device 1000 described above may be stored in the memory 1200 of the electronic device 1000, and the processor 1300 of the electronic device 1000 may perform the operations stored in the memory 1200.

The features, structures, effects, and the like described in the above embodiments are included in at least one embodiment of the present invention and are not necessarily limited to only one embodiment. Further, the features, structures, effects, and the like illustrated in each embodiment can be combined or modified for implementation in other embodiments by those of ordinary skill in the art to which the embodiments pertain. Accordingly, the content related to such combinations and modifications should be construed as falling within the scope of the present invention.

Although embodiments have been mainly described above, these are merely illustrative and do not limit the present invention. Those skilled in the art to which the present invention pertains should understand that several modifications and applications that have not been described above can be made without departing from the fundamental characteristics of the present embodiments. In other words, each component specifically presented in the embodiments may be modified for implementation. In addition, differences associated with these modifications and applications are to be construed as falling within the scope of the present invention defined by the appended claims.

1: A method of calculating an expected adult height by an electronic device, the method comprising:
generating a growth prediction model; and
calculating an expected adult height using the generated growth prediction model,
wherein the generating of the growth prediction model comprises:
acquiring growth statistics data and input data including height information at a time of measurement, chronological age information at the time of measurement, bone age information at the time of measurement, and sex information;
calculating an adult height on the basis of the input data and the growth statistics data;
acquiring an actual adult height of a child corresponding to the input data; and
generating the growth prediction model on the basis of the input data, the calculated adult height, and the actual adult height.

2: The method of claim 1, wherein the calculating of the adult height further comprises:
acquiring a first percentile corresponding to the chronological age information and the height information of the input data from the growth statistics data on the basis of the height information and the chronological age information; and
calculating a first height corresponding to the first percentile as the adult height using growth statistics data of a group of people whose growth has stopped.

3: The method of claim 1, wherein the calculating of the adult height further comprises:
acquiring a second percentile corresponding to the bone age information and the height information of the input data from the growth statistics data on the basis of the height information and the bone age information; and
calculating a second height corresponding to the second percentile as the adult height using growth statistics data of a group of people whose growth has stopped.

4: The method of claim 2, wherein the generating of the growth prediction model further comprises:
inputting the height information, the chronological age information, the bone age information, and the sex information of the input data and the calculated adult height as input values for the growth prediction model;
inputting the actual adult height as an output value of the growth prediction model; and
calculating a weight of the growth prediction model for outputting the output value from the input values.

5: The method of claim 4, wherein the calculating of the expected adult height further comprises:
acquiring subject input data including height information at a time of analysis, chronological age information at the time of analysis, bone age information at the time of analysis, and sex information regarding a subject whose expected adult height will be analyzed and growth statistics data of a group to which the subject belongs; and
calculating the expected adult height from the subject input data using the growth prediction model including the calculated weight.

6: The method of claim 5, wherein the calculating of the expected adult height further comprises:
calculating an adult height from the subject input data of the subject on the basis of the growth statistics data; and
inputting the subject input data and the calculated adult height into the growth prediction model and acquiring the expected adult height output from the growth prediction model.

7: The method of claim 6, wherein the generating of the growth prediction model further comprises:
identifying a preset age band on the basis of the chronological age information or the bone age information of the subject input data; and
when an identified age band corresponds to a first band, generating a first model including a first weight on the basis of a dataset composed of adult heights which are calculated from the growth statistics data and input data corresponding to the first band, and the actual adult height, and when the identified age band corresponds to a second band, generating a second model including a second weight on the basis of a dataset of adult heights which are calculated from the growth statistics data and input data corresponding to the second band, and the actual adult height.

8: The method of claim 7, wherein the calculating of the expected adult height further comprises:
identifying a preset age band on the basis of the chronological age information or the bone age information of the subject input data; and
when the identified age band corresponds to the first band, calculating the expected adult height from the adult height calculated from the subject input data and the growth statistics data using the first model, and when the identified age band corresponds to the second band, generating the expected adult height from the adult height calculated from the subject input data and the growth statistics data using the second model.

9: A computer-readable recording medium on which a program for causing a computer to execute the method of claim 1 is recorded.

10: An electronic device for predicting an adult height, comprising a processor configured to acquire growth statistics data and input data including height information at a time of measurement, chronological age information at the time of measurement, bone age information at the time of measurement, and sex information, calculate an adult height on the basis of the input data and the growth statistics data, acquire an actual adult height of a child corresponding to the input data, generate a growth prediction model on the basis of the input data, the calculated adult height, and the actual adult height, and calculate an expected adult height using the generated growth prediction model.
